(19) United States
(12) Patent Application Publication
(10) Pub. No.: US 2008/0161653 A1
(43) Pub. Date: Jul. 3, 2008

(54) CARDIO-RESPIRATORY FITNESS EVALUATION METHOD AND SYSTEM

(75) Inventors: Ming-Hui Lin, Hsinchu County (TW); Shang-Yuan Cheng, Hsinchu City (TW); Tung-Wu Lu, Taipei County (TW)

Correspondence Address: QUINTERO LAW OFFICE, PC, 2210 MAIN STREET, SUITE 200, SANTA MONICA, CA, 90405, US

(73) Assignee: INDUSTRIAL TECHNOLOGY RESEARCH INSTITUTE, HSINCHU (TW)

(21) Appl. No.: 11/735,927

(22) Filed: Apr. 16, 2007

(30) Foreign Application Priority Data
Dec. 29, 2006 (TW) TW95149882

(51) Int. Cl. A61B 5/00; A61B 5/083

(52) U.S. Cl. 600/300; 600/301

(57) ABSTRACT

A cardio-respiratory fitness evaluation method is disclosed. Personal data is obtained according to an input operation and a submaximal exercise test is performed according to the personal data and a selected exercise mode. Physical parameters are retrieved and analyzed to determine whether abnormal signs are detected and, if not, the physical parameters are recorded. It is then determined whether the submaximal exercise test has been completed, and, if not completed, another submaximal exercise test is performed, and if completed, an evaluation for cardio-respiratory fitness is performed to obtain evaluation results.

### BACKGROUND OF THE INVENTION

1. Field of the Invention

The invention relates to a data evaluation system, and more particularly to a cardio-respiratory fitness evaluation method and system.

2. Description of the Related Art

Current fitness equipment, such as exercise bikes and treadmills, typically only provide internally installed user selectable training routines, but these training routines are not based on individual physical and cardio-respiratory fitness. A user may engage in excess, needless, or insufficient exercise to the limited number of training routines provided by conventional fitness equipment.

The measurement of oxygen consumption comprises measuring and prediction methods. The measuring method gathers breath using a Douglas Bag to measure the oxygen difference of breathing in and out within a predetermined time period and calculates measurements of oxygen content per minute based on carbon dioxide analysis. The prediction method indirectly predicts measurements of oxygen content using fitness equipment in conjunction with physical indexes, such as heart rates.

U.S. Pat. No. 7,054,678 discloses a system and method for assessing and modifying the physiological conditions of individuals. Cycle and shape parameters are derived from a recorded time trace containing heart rate data collected while an individual performs a cyclic exercise routine. Individually tailored exercise regimens that are based on these parameters are generated and modified as desired.

T.W. Patent No. 357077 discloses a device for supporting exercise recipes, providing a maximum oxygen derivation device. Exercise strength is calculated according to recorded heart rates, steps, and step breadths. The maximum oxygen content is evaluated according to the maximum oxygen consumption relationship between pre-stored physical exertion and heart rates. When a linear relationship between heart rate and physical exertion is not detected, the maximum oxygen content must be re-measured, which is inconvenient.

Thus, the invention provides a cardio-respiratory fitness evaluation method and system, obtaining personal physical parameters for analysis to obtain the maximum oxygen consumption (VO2max) to evaluate personal cardio-respiratory fitness, thereby calculating a functional aerobic impairment (FAI) value to be referred to exercise recipes.

### BRIEF SUMMARY OF THE INVENTION

Cardio-respiratory fitness evaluation methods are provided. An exemplary embodiment of a cardio-respiratory fitness evaluation method comprises the following. Personal data is obtained according to an input operation and a submaximal exercise test is performed according to the personal data and a selected exercise mode. Physical parameters are retrieved and analyzed to determine whether abnormal signs are detected and, if not, the physical parameters are recorded. It is then determined whether the submaximal exercise test has been completed, and, if not, another submaximal exercise test is performed, and if so, an evaluation for cardio-respiratory fitness is performed to obtain evaluation results.

Cardio-respiratory fitness evaluation systems are provided. An exemplary embodiment of a cardio-respiratory fitness evaluation system comprises a calculation device and a motion device. The calculation device further comprises an input unit obtaining personal data and an exercise mode, a memory unit storing the personal data, a process unit, and an evaluation unit. The motion device performs a submaximal exercise test according to the personal data and the exercise mode. The process unit retrieves personal physical parameters for analysis, determines whether abnormal signs are detected according to the analysis result. If abnormal signs are detected, the test is terminated, if not, the personal physical parameters are recorded, and it is determined whether the submaximal exercise test has been completed. If not completed another submaximal exercise test is performed. If completed, the evaluation unit performs an evaluation for cardio-respiratory fitness to obtain an evaluation result.

A detailed description is given in the following with reference to the accompanying drawings.

### BRIEF DESCRIPTION OF THE DRAWINGS

The invention can be more fully understood by reading the subsequent detailed description and examples with references made to the accompanying drawings, wherein:

FIG. 1 is a schematic view of an embodiment of a cardio-respiratory fitness evaluation system;

FIG. 2 is a flowchart of an embodiment of a cardio-respiratory fitness evaluation method;

FIG. 3 is a schematic view of an embodiment of motion device 1200 shown in FIG. 1;

FIG. 4 is a schematic view of an embodiment of memory unit 1120 shown in FIG. 1; and

FIG. 5 is a flowchart of an embodiment of a cardio-respiratory fitness evaluation method, evaluating personal maximum oxygen consumption (VO2max).

### DETAILED DESCRIPTION OF THE INVENTION

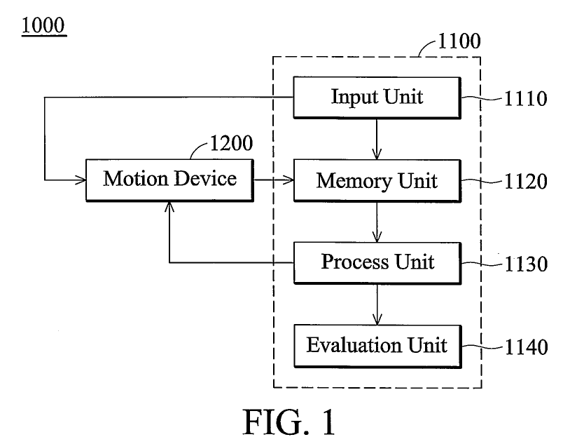

Several exemplary embodiments of the invention are described with reference to FIGS. 1 through 5, which generally relate to cardio-respiratory fitness evaluation. It is to be understood that the following disclosure provides various embodiments as examples for implementing different features of the invention. Specific examples of components and arrangements are described in the following to clearly illustrate the disclosed. These are, of course, merely examples and are not intended to be limiting. In addition, the reference numerals and/or letters may be repeated where applicable in the various examples. This repetition is for the purpose of simplicity and clarity and does not in itself dictate a relationship between the various described embodiments or configurations.

The invention discloses a cardio-respiratory fitness evaluation method and system.

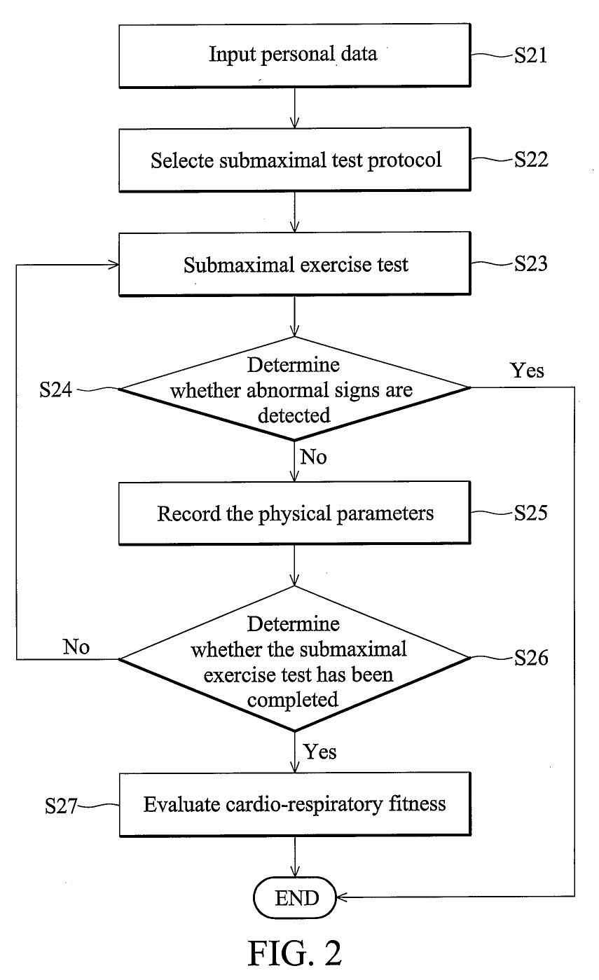

FIG. 1 is a schematic view of an embodiment of a cardio-respiratory fitness evaluation system. FIG. 2 is a flowchart of an embodiment of a cardio-respiratory fitness evaluation method, evaluating personal cardio-respiratory fitness.

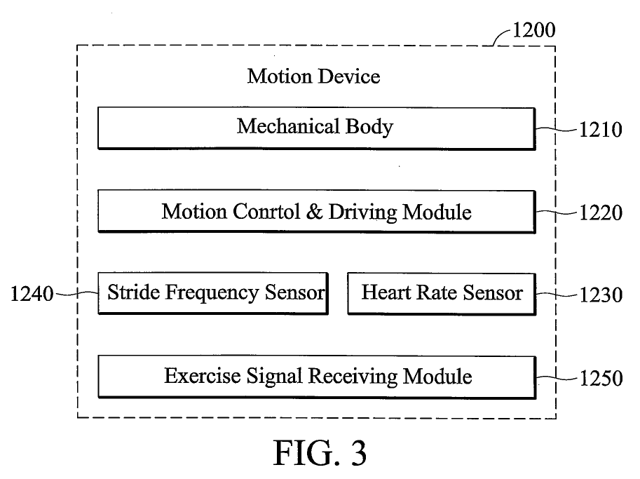

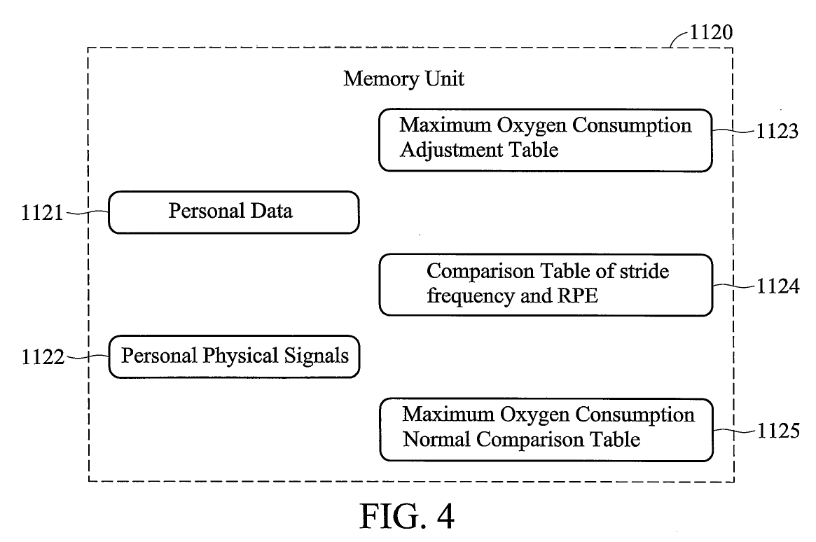

Cardio-respiratory fitness evaluation system 1000 comprises a calculation device 1100 and a motion device 1200. Calculation device 1100 further comprises an input unit 1110, a memory unit 1120, a process unit 1130, and an evaluation unit 1140. Motion device 1200 further comprises a mechanical body 1210, an motion control and driving module 1220, a heart rate sensor 1230, a stride frequency sensor 1240, and an exercise signal receiving module 1250, as shown in FIG. 3. Memory unit 1120 stores personal data 1121, personal physical signals 1122, maximum oxygen consumption adjustment table 1123, and a comparison table of stride frequency and rate of perceived exertion (RPE) 1124, and a maximum oxygen consumption normal comparison table 1125, as shown in FIG. 4.

Referring to FIGS. 1 and 2, personal data, comprising age, height, weight, sex, and so forth, is first input by input unit 1110 (step S21) and stored in memory unit 1120 (personal data 1121). A submaximal exercise test protocol is selected using input unit 1110 (step S22). The maximum oxygen consumption (VO2max), for example, could be calculated only if the user follows instructions of a treadmill test protocol.

Next, motion control and driving module 1220 drives mechanical body 1210 according to the input personal data and the selected submaximal exercise test mode to perform a submaximal exercise test (step S23). Motion device 1200, such as an exercise bike capable of setting gradients and speeds, provides different and recurring workloads. Heart rate sensor 1230 and stride frequency sensor 1240 obtains physical signals (personal physical signals 1122) during the submaximal exercise test. Stride frequency sensor 1240 may be an accelerometer, detecting stride frequency (i.e. fatigue strength) by recording acceleration and time, then quantifies and classifies stride delays, replacing subjective perceived exertion.

Process unit 1130 obtains and analyzes the physical parameters to determine whether abnormal signs are detected while exercising (step S24). The resulting relationship between heart rates and workload, for example, does not represent a linear relationship due to physical fatigue. If abnormal signs are detected, the process terminates. If no abnormal sign is detected, process unit 1130 records the physical parameters that exercise signal receiving module 1250 transmits and stores the physical signals, retrieved from heart rate sensor 1230 and a stride frequency sensor 1240, in memory unit 1120 (step S25).

Next, process unit 1130 determines whether the submaximal exercise test has been completed (step S26). If not completed, another submaximal exercise test is performed. If completed, evaluation unit 1140 performs an evaluation for cardio-respiratory fitness to obtain an evaluation result (step S27), and the process terminates.

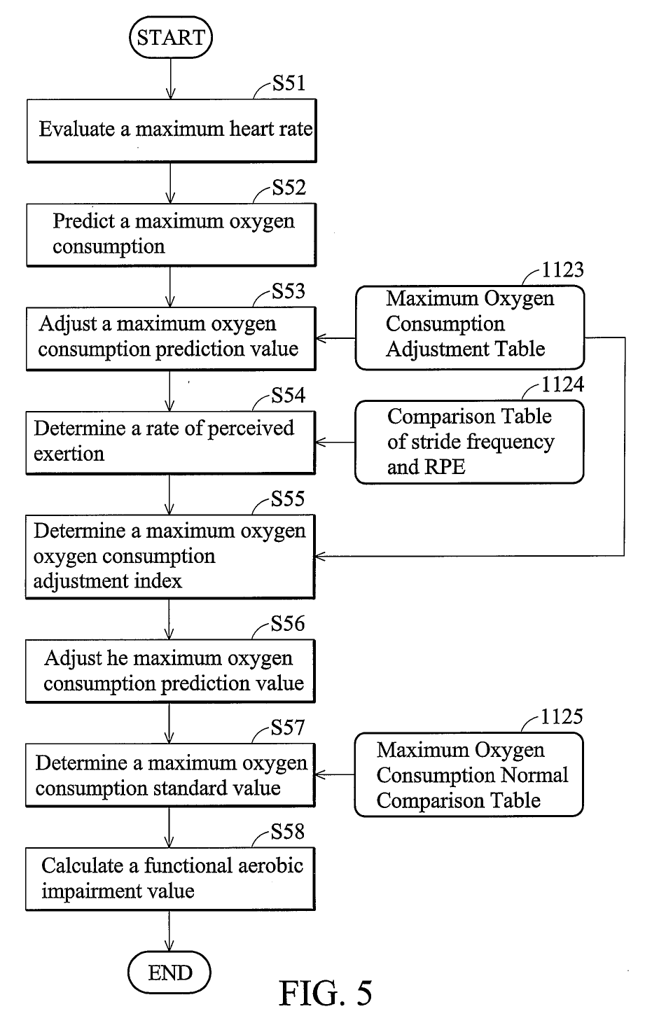

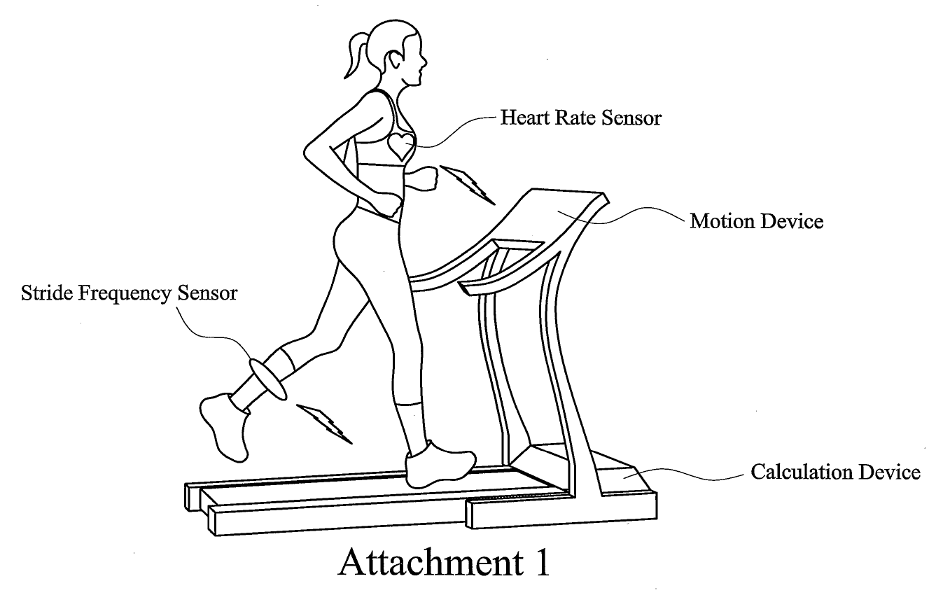

When the submaximal exercise test is complete, maximum oxygen consumption (VO2max) is evaluated. FIG. 5 is a flowchart of an embodiment of a cardio-respiratory fitness evaluation method, evaluating personal maximum oxygen consumption (VO2max).

A maximum heart rate is first evaluated according to the personal data (step S51). A maximum oxygen consumption is predicted according a selected submaximal exercise mode (step S52). The maximum oxygen consumption prediction value is first adjusted according to the corresponding maximum heart rate included in maximum oxygen consumption adjustment table 1123 (step S53).

Stride frequencies are analyzed and a rate of perceived exertion is determined according to a stride frequency and rate of perceived exertion (RPE) 1124 (step S54) comparison table. The stride frequency and rate of perceived exertion (RPE) 1124 comparison table can be experimentally, statistically, and summarily generated. Next, determine a maximum oxygen consumption adjustment index (step S55) according to the rate of perceived exertion included in the maximum oxygen consumption adjustment table 1123 (step S53). Next, the first adjusted maximum oxygen consumption prediction value is adjusted again according to the maximum oxygen consumption adjustment index to obtain a maximum oxygen consumption evaluation value.

A maximum oxygen consumption standard value is known when check the maximum oxygen consumption normal comparison table 1125 (step S57). A functional aerobic impairment (FAI) value is calculated by comparing a maximum oxygen consumption evaluation value with a standard value. (step S58). Thus, when compared with normal modes for contemporary and the same sex groups, the difference can be obtained for exercise recipe reference.

Attachment 1 illustrates an exercise process using an exercise bike based on an embodiment of a cardio-respiratory fitness evaluation method. A stride frequency sensor can be a three-dimensional (3D) accelerometer. A calculation device can be an independent personal computer or an embedded single chip. When a user exercises, a heart rate sensor and the stride frequency sensor repeatedly obtains physical parameters of the exerciser and transmits the signals using a wired or wireless method to the calculation device for analysis and processing, thus the difference can be obtained for exercise recipe reference.

An embodiment of a cardio-respiratory fitness evaluation method changes workload based on modulation parameters provided by, but not limited to, a programmable control motion device, such as an exercise bike capable of setting gradients and speeds that provides recurring levels physical exertion. The maximum oxygen consumption (VO2max) is evaluated according to oxygen consumption (VO2), and a linear relationship between heart rates and workload to determine an exercise recipe.

Further, it is noted that the method and system can be integrated with a portable exercise device, which can be worn by a user. The portable exercise device retrieves physical signals of the user, emits audio signals when adjusting exercise strength, and calculates the maximum oxygen consumption (VO2max) according to exercise mode, thereby determining an exercise recipe corresponding to oxygen consumption (VO2).

Methods and systems of the present disclosure, or certain aspects or portions of embodiments thereof, may take the form of program code (i.e., instructions) embodied in media, such as floppy diskettes, CD-ROMS, hard drives, firmware, or any other machine-readable storage medium, wherein, when the program code is loaded into and executed by a machine, such as a computer, the machine becomes an apparatus for practicing embodiments of the disclosure. The methods and apparatus of the present disclosure may also be embodied in the form of program code transmitted over some transmission medium, such as electrical wiring or cabling, through fiber optics, or via any other form of transmission, wherein, when the program code is received and loaded into and executed by a machine, such as a computer, the machine becomes an apparatus for practicing and embodiment of the disclosure. When implemented on a general-purpose processor, the program code combines with the processor to provide a unique apparatus that operates analogously to specific logic circuits.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A cardio-respiratory fitness evaluation method, comprising:
performing a submaximal exercise test and an analysis operation according to personal data and an exercise mode;
determining whether abnormal signs are detected according to an analysis result;
if detected, terminating the test;
if not detected, recording personal physical parameters; and
when the motion test is complete, performing an evaluation for cardio-respiratory fitness to obtain an evaluation result.

2. The cardio-respiratory fitness evaluation method as claimed in claim 1, further comprising:
obtaining the personal data according to an input operation;
performing the submaximal exercise test and the analysis operation according to the personal data and a selected exercise mode;
when no abnormal signs are detected and the submaximal exercise test has not been completed, performing another submaximal exercise test to obtain the evaluation result.

3. The cardio-respiratory fitness evaluation method as claimed in claim 1, wherein recording the personal physical parameters obtains personal physical signals using a heart rate sensor and a stride frequency sensor and transmits and stores the signals in a memory unit using a wired or wireless method.

4. The cardio-respiratory fitness evaluation method as claimed in claim 1, further comprising:
evaluating a maximum heart rate according to the personal data;
obtaining a maximum oxygen consumption prediction value according the selected submaximal exercise mode;
adjusting the maximum oxygen consumption prediction value according to the corresponding maximum heart rate;
analyzing stride frequencies and determining a rate of perceived exertion according to a stride frequency and a comparison table of rate of perceived exertion;
determining a maximum oxygen consumption adjustment index according to the rate of perceived exertion;
adjusting the maximum oxygen consumption prediction value according to the maximum oxygen consumption adjustment index to obtain a maximum oxygen consumption evaluation value;
determining a maximum oxygen consumption standard value according to a maximum oxygen consumption normal comparison table; and
calculating a functional aerobic impairment value by comparing a maximum oxygen consumption evaluation value with a standard value.

5. The cardio-respiratory fitness evaluation method as claimed in claim 1, further comprising adjusting motion loading according to modulation parameters provided by a programmable control motion device to obtain another evaluation result, wherein the motion strength of the programmable control motion device is recurrent.

6. The cardio-respiratory fitness evaluation method as claimed in claim 1, further comprising evaluating the maximum oxygen consumption according to oxygen consumption, heart rates, and its linear relationship relating to workload output of the motion device.

7. A cardio-respiratory fitness evaluation system, comprising:
a calculation device, further comprising:
an input unit, obtaining personal data and an exercise mode;
a memory unit, storing the personal data;
a process unit; and
an evaluation unit; and
a motion device, performing a submaximal exercise test according to the personal data and the exercise mode;
wherein the process unit retrieves personal physical parameters for analysis, determines whether abnormal signs are detected according to the analysis result, if detected, terminates the test, if not detected, records the personal physical parameters, determines whether the motion test has been completed, and, if not completed, performs another submaximal exercise test, and, if completed, the evaluation unit performs an evaluation for cardio-respiratory fitness to obtain an evaluation result.

8. The cardio-respiratory fitness evaluation system as claimed in claim 7, further comprising a heart rate sensor and a stride frequency sensor, wherein the process unit obtains personal physical signals using the heart rate sensor and the stride frequency sensor and transmits and stores the signals in the memory unit using a wired or wireless method.

9. The cardio-respiratory fitness evaluation system as claimed in claim 7, wherein the process unit further evaluates a maximum heart rate according to the personal data, obtains a maximum oxygen consumption prediction value according the selected submaximal exercise mode, adjusts the maximum oxygen consumption prediction value according to the corresponding maximum heart rate, analyzes stride frequencies and determining a rate of perceived exertion according to a stride frequency and a comparison table of rate of perceived exertion, determines a maximum oxygen consumption adjustment index according to the rate of perceived exertion, adjusts the maximum oxygen consumption prediction value according to the maximum oxygen consumption adjustment index to obtain a maximum oxygen consumption evaluation value, determines maximum oxygen consumption standard value according to a maximum oxygen consumption normal comparison table, and calculates functional aerobic impairment value by comparing a maximum oxygen consumption evaluation value with a standard value.

10. The cardio-respiratory fitness evaluation system as claimed in claim 7, wherein the motion device is a programmable control motion device, adjusting motion loading according to modulation parameters to obtain another evaluation result, wherein the motion strength of the programmable control motion device is recurrent.

11. The cardio-respiratory fitness evaluation system as claimed in claim 7, wherein the process unit further evaluates the maximum oxygen consumption according to oxygen consumption, heart rates, and its linear relationship relating to workload output of the motion device.
